(19) United States
(12) Patent Application Publication
(10) Pub. No.: US 2012/0100187 A1
(43) Pub. Date: Apr. 26, 2012

(54) COATINGS AND METHODS FOR CONTROLLED ELUTION OF HYDROPHILIC ACTIVE AGENTS

(75) Inventors: Ralph A. Chappa, Ham Lake, MN (US); Michael J. Burkstrand, Richfield, MN (US); David E. Babcock, St. Louis Park, MN (US)

(73) Assignee: SURMODICS, INC., Eden Prairie, MN (US)

(21) Appl. No.: 13/281,802

(22) Filed: Oct. 26, 2011

Related U.S. Application Data
(60) Provisional application No. 61/406,906, filed on Oct. 26, 2010.

(51) Int. Cl. A61K 9/00; A61K 31/7052; B05D 5/00; A61K 38/02

(52) U.S. Cl. 424/400; 514/1.1; 514/44 R; 427/2.21

(57) ABSTRACT

Embodiments of the invention include multi-layer coatings and methods for controlling the elution of hydrophilic active agents. In an embodiment, the invention includes a medical device including a substrate, a primer polymer layer disposed on the substrate, an expandable layer disposed on the primer polymer layer, and a hydrophilic active agent dispersed within the expandable layer. In an embodiment, the invention includes a method of forming a medical device including depositing a primer layer onto a substrate, the primer layer comprising a primer polymer. The method can further include depositing an expandable layer onto the primer layer, the expandable layer including an expandable polymer, and a hydrophilic active agent. The expandable layer can be deposited with a solvent that is effective to solvate both the expandable polymer and the primer polymer. Other embodiments are also included herein.

This application claims the benefit of U.S. Provisional Application No. 61/406,906, filed Oct. 26, 2010, the content of which is herein incorporated by reference in its entirety.

### FIELD OF THE INVENTION

The present invention relates to coatings and methods for controlling the elution of hydrophilic active agents.

### BACKGROUND OF THE INVENTION

Active agent elution control coatings are now commonly used to deliver active agents to tissues of the body. Elution control coatings can enable the delivery of an active agent over a period of time in order to optimize therapeutic effect. In addition, when disposed on a medical device, elution control coatings can enable site-specific active agent delivery because the medical device can be positioned as desired within the body of a patient.

Active agents delivered from elution control coatings can include many different types of compounds including small hydrophilic molecules, small hydrophobic molecules, hydrophilic macromolecules such as carbohydrates, peptides, proteins, and the like.

Frequently, the usefulness of an elution control system can depend on its ability to release an active agent at a therapeutically desirable rate. Releasing an active agent too fast or too slow may prevent the active agent from achieving the desired therapeutic effect.

Accordingly, there is a need for coatings that can deliver active agents at desirable rates and methods of making the same.

### SUMMARY OF THE INVENTION

Embodiments of the invention include multi-layer coatings and methods for controlling the elution of hydrophilic active agents. In an embodiment, the invention includes a medical device including a substrate, a primer polymer layer disposed on the substrate, an expandable layer disposed on the primer polymer layer, and a hydrophilic active agent dispersed within the expandable layer.

In an embodiment, the invention includes a method of forming a medical device including depositing a primer layer onto a substrate, the primer layer comprising a primer polymer. The method can further include depositing an expandable layer onto the primer layer, the expandable layer including an expandable polymer, and a hydrophilic active agent. The expandable layer can be deposited with a solvent that is effective to solvate both the expandable polymer and the primer polymer.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a schematic view of a medical device in accordance with an embodiment of the invention.

FIG. 2 is a cross-sectional view of the medical device of FIG. 1, as taken along line 2-2′.

FIG. 3 is a cross-sectional view of a medical device in accordance with another embodiment.

FIG. 4 is a cross-sectional view of a coating in accordance with another embodiment.

FIG. 5 is a schematic view of a medical device in accordance with an embodiment of the invention.

FIG. 6 is a cross-sectional view of a portion of the medical device shown in FIG. 5.

FIG. 7 is a schematic view of a medical device in accordance with an embodiment of the invention.

FIG. 8 is a cross-sectional view of a portion of the medical device shown in FIG. 7.

FIG. 9 is a graph of cumulative Fab elution from various coating configurations over time.

FIG. 10 is a graph of water uptake over time with various coating configurations over time.

FIG. 11 is a graph of cumulative percent Fab elution over time from coatings with various topcoats.

FIG. 12 is a graph of water uptake over time from coatings with various topcoats.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

As described above, active agents delivered from elution control coatings can include many different types of compounds including small hydrophilic molecules, small hydrophobic molecules, hydrophilic macromolecules such as carbohydrates, peptides, proteins, and the like. Of these agents, hydrophilic agents, in general, can pose a challenge in controlled elution systems because they are more difficult to elute at a desirable rate, tending to elute either too fast or too slow.

In some cases where elution of hydrophilic agents is too fast, it is believed that the fast elution rate is at least partially a result of a build-up of fluid pressure inside of the layer carrying the active agent. While not intending to be bound by theory, it is believed that such a build-up of pressure acts to force the hydrophilic active agent out of the coating more quickly than would otherwise occur.

In various embodiments herein, a multi-layered coating is provided that can elute hydrophilic active agents at desirable rates. In such embodiments, the active agent can be disposed within a layer that can physically expand. It is believed that allowing the layer to physically expand can reduce and/or prevent a build-up in pressure that may otherwise occur due to the ingress of fluids into the layer. As a result, the active agent can be released at a more desirable rate. In an embodiment, a medical device is included having a substrate, a primer polymer layer disposed on the substrate, and an expandable layer disposed on the primer polymer layer. A hydrophilic active agent can be dispersed within the expandable layer. In some embodiments, a top layer can be disposed over the expandable layer. The top layer can be configured to allow the expandable layer to expand.

Referring now to FIG. 1, a schematic view is shown of a medical device 100 in accordance with an embodiment of the invention. In this embodiment, the medical device 100 is an eye screw. However, it will be appreciated that other types of medical device are also included within the scope herein. Further examples of medical devices are described below. The medical device 100 includes a tip 102, a coiled body 104, and a cap member 106.

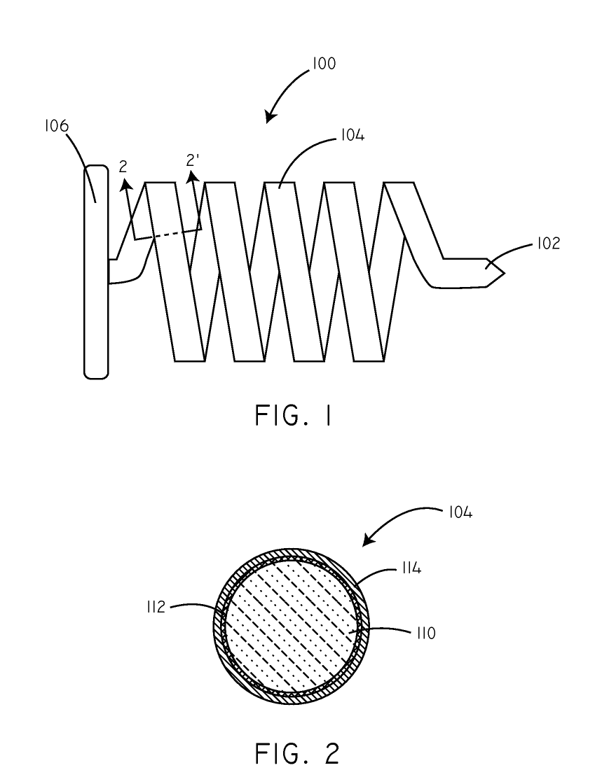

Referring now to FIG. 2, a cross-sectional view of the medical device 100 of FIG. 1 is shown as taken along line 2-2′ of FIG. 1. In this view, a primer layer 112 (or non-expandable layer) is disposed on a substrate 110. While not intending to be bound by theory, it is believed that the primer layer 112 can be used to improve adhesion of the entire coating to the substrate. The substrate 110 can include various materials as described more fully below, including but not limited to, metals, ceramics, polymers, glasses, and the like.

The primer layer 112 can include one or more polymers. In some embodiments, the primer layer 112 can be substantially non-expandable. For example, the primer layer 112 can be comprised of materials such that the primer layer 112 expands less than about 5 percent in volume after insertion into a patient. One or more primer layers can be included. In some embodiments, multi-layer elution control coatings can include a first primer layer and a second primer layer, the second either the same or different than the first.

Exemplary polymers used to form the primer layer 112 can include poly-n-butyl methacrylate (PBMA), polystyrene, and other polymers that are substantially non-expandable in an aqueous environment.

An expandable layer 114 (or active agent layer) can be disposed on the primer layer 112. In contrast to the primer layer 112, the expandable layer 114 can be expandable. As used herein, the term “expandable” shall mean that the layer can expand in volume, such as after insertion in vivo. In various embodiments, the expandable layer 114 can expand in volume greater than about 5 percent after insertion into a patient. The expandable layer 114 can include a material that can expand (polymeric or non-polymeric) and can also include one or more hydrophilic active agents. In general, expansion of the expandable layer can be triggered by the ingress of fluid, such as the ingress of an aqueous fluid after the device has been implanted within a patient.

Exemplary materials used to form the expandable layer can include those that are expandable. By way of example, exemplary materials used to form the expandable layer can include hydrophobic polymers, hydrophilic polymers, degradable polymers, non-polymeric expandable materials, and the like.

Hydrophobic polymers used in accordance with embodiments herein can include both elastomeric and non-elastomeric polymers. Exemplary hydrophobic polymers can specifically include polyethylene-co-vinyl acetate (PEVA) (such as PEVA including 33 wt. % vinyl acetate content and PEVA including 40 wt. % vinyl acetate content), cross-linked polysiloxane (silicone rubber), butyl rubber, styrene-butadiene rubber, polybutadiene, ethylene-propylene, polychloroprene, polyisoprene, nitrile rubber, urethane rubber, cross-linked forms of the same, and the like.

Hydrophilic polymers used in accordance with embodiments herein can include both elastomeric and non-elastomeric polymers. Hydrophilic polymers used in accordance with embodiments herein can specifically include hydrogels, polylactide-co-glycolide (PLGA), polyvinylpyrrolidone (PVP) and co-polymers including PVP, biodegradable polymers, and the like. Hydrogels can include both natural and synthetic polymers. Hydrogels can include neutral hydrogels, anionic hydrogels, cationic hydrogels, and ampholytic hydrogels. Hydrogels can specifically include hyaluronic acid, alginic acid, pectin, carrageenana, chrondroitin sulfate, dextran sulfate, chitosan, polylysine, collagen, gelatin, carboxymethyl chitin, fibrin, dextran, agarose, pullulan, polyesters, polyethylene glycol (PEG) copolymers (such as PEG-PLA-PEG, PEG-PLGA-PEG, PEG-PCL-PEG, PLA-PEG-PLA), PEG/PBO terephthalate, PEG-bis-(PLA-acrylate), poly(PEG-co-peptides), PEG-g-P(AAm-co-Vamine), PAAm, poly(NIPAAm-co-AAc), poly(NIPAAm-co-EMA), PVAc/PVA, PNVP, poly(MMA-co-HEMA), poly(AN-co-allyl sulfonate), poly(biscarboxyphenoxy-phosphazene), poly(GEMA-sulfate), alginate-g-(PEO-PPO-PEO), poly(PLGA-co-serine), collagen acrylate, alginate-acrylate, poly(HPMA-g-peptide), HA-g-NIPAAm, polyvinylpyrrolidone, cross-linked forms of the same, and the like.

Polymers used in conjunction with various embodiments herein can also include both natural and synthetic degradable polymers. Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), polypropylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and copolymers thereof. Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)-]y

where —C₆H₄— designates the divalent aromatic ring residue from each esterified molecule of terephthalic acid, n represents the number of ethylene oxide units in each hydrophilic

PEG block, x represents the number of hydrophilic blocks in the copolymer, and y represents the number of hydrophobic blocks in the copolymer. The subscript “n” can be selected such that the molecular weight of the PEG block is between about 300 and about 4000. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure. Such degradable polymers can specifically include those described in U.S. Pat. No. 5,980,948, the content of which is herein incorporated by reference in its entirety.

Degradable polyesteramides can include those formed from the monomers OH-x-OH, z, and COOH-y-COOH, wherein x is alkyl, y is alkyl, and z is leucine or phenylalanine. Such degradable polyesteramides can specifically include those described in U.S. Pat. No. 6,703,040, the content of which is herein incorporated by reference in its entirety.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

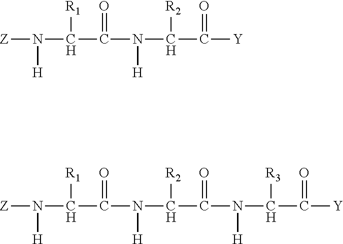

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, α aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Publ. Pat. Application No. 20070218102, entitled “HYDROPHOBIC DERIVATIVES OF NATURAL BIODEGRADABLE POLYSACCHARIDES”, all of which are herein incorporated by reference in their entirety.

Degradable polymers of the invention can also include dextran based polymers such as those described in U.S. Pat. No. 6,303,148, entitled “PROCESS FOR THE PREPARATION OF A CONTROLLED RELEASE SYSTEM”, the content of which is herein incorporated by reference in its entirety. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides(oxepane-2,7-dione). The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

Non-polymeric expandable materials can include materials that are pliant and expand. Specific examples of non-polymeric expandable materials can specifically include petroleum jelly, silicone vacuum grease, and the like.

In some embodiments, the active agent is dispersed within the expandable layer 114. As used herein, the term “dispersed” shall refer to the property of being distributed in a soluble or insoluble state throughout the layer.

As used herein, the term “active agent” means a compound that has a particular desired activity. For example, an active agent can be a therapeutic compound that exerts a specific activity on a subject. Exemplary hydrophilic active agents can include peptides, proteins, antibodies, antibody fragments, carbohydrates, nucleic acids, lipids, polysaccharides, synthetic inorganic or organic molecules, or combinations thereof that cause a desired biological effect when administered to an animal, including but not limited to birds and mammals, including humans. Hydrophilic active agents used with the invention can specifically include proteins, protein fragments, peptides, polypeptides, and the like. Peptides can include any compound containing two or more amino-acid residues joined by amide bonds formed from the carboxyl group of one amino acid and the amino group of the next one. By way of example, peptides can include glycosylated proteins, antibodies (both monoclonal and polyclonal), antibody derivatives (including diabodies, f(ab) fragments, humanized antibodies, etc.), cytokines, growth factors, receptor ligands, enzymes, and the like. Hydrophilic active agents used with embodiments herein can also include, but are not limited to, antibiotics such as gentamycin and tobramycin, amongst others. Hydrophilic active agents used with embodiments herein can also include those with anti-inflammatory activity including, but not limited to, dexamethasone phosphate.

The primer layer 112 can be deposited onto the substrate 110 using any of a variety of coating techniques including dip-coating, spray-coating (including both gas-atomization and ultrasonic atomization), fogging, brush coating, press coating, blade coating, and the like. The primer layer 112 may be applied as a coating solution and may be applied under conditions where atmospheric characteristics such as relative humidity, temperature, gaseous composition, and the like are controlled. In some embodiments, the coating solution is applied using a spray technique. Exemplary spray coating equipment that can be used to apply components of the invention can be found in U.S. Pat. No. 6,562,136; U.S. Pat. No. 7,077,910; U.S. Pub. App. No. US 2004/0062875; U.S. Pub. App. No. 2005/0158449; U.S. Pub. App. No. 2006/0088653; U.S. Pub. App. No. 2005/0196424; and U.S. Pub. App. No. 2007/0128343, the contents of which are all hereby incorporated by reference.

Similarly, the expandable layer 114 can be deposited on the primer layer 112 as a solution and can be deposited using techniques such as dip-coating, spray-coating (including both gas-atomization and ultrasonic atomization), fogging, brush coating, press coating, blade coating, and the like.

Various solvents can be used in order to form coating solutions for deposition of the primer layer 112, expandable layer 114, and/or top layer 116. Solvents can include both polar and non-polar solvents. Solvents can include water, alcohols (e.g., methanol, butanol, propanol, and isopropanol (isopropyl alcohol)), alkanes (e.g., halogenated or unhalogenated alkanes such as chloroform, hexane, and cyclohexane), amides (e.g., dimethylformamide), ethers (e.g., THF and dioxolane), ketones (e.g., acetone, methylethylketone), aromatic compounds (e.g., toluene and xylene), nitriles (e.g., acetonitrile) and esters (e.g., ethyl acetate).

In some embodiments, a solvent is chosen for use in preparing a solution to deposit the expandable layer 114 that can also be effective for solvation of the polymer(s) of the primer layer 112. For example, the selected solvent can not only serve as a solvent for the polymer used in the expandable layer 114, but can also solvate the polymer of the primer layer 112. While not intending to be bound by theory, it is believed that where the polymer of primer layer 112 is solvated during deposition of the expandable layer 114 there is greater adherence between the expandable layer 114 and the primer layer 112.

In some embodiments, such as where the substrate includes a polymer that is solvated by the solvent used to deposit the expandable layer, the primer layer can be omitted.

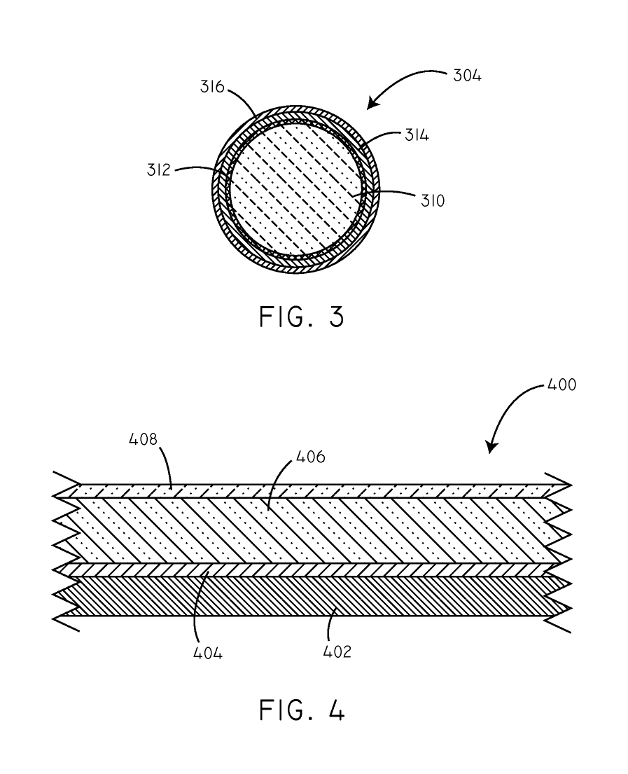

In some embodiments a top layer (or top coat) can be disposed on the expandable layer. Referring now to FIG. 3 a cross-sectional view is shown of a coating 304 including a top layer. In this embodiment, a primer layer 312 is disposed on top of a substrate 310. Further, an expandable layer 314 including an active agent is disposed on top of the primer layer 312. Finally, a top layer 316 is disposed on top of the expandable layer 314. The top layer 316 can be configured to stretch as the expandable layer 314 swells. The top layer 316 can further modulate release of the hydrophilic active agent. The top layer 316 can include polymers that allow the expandable layer to expand. An exemplary polymer for the top layer 316 can include polyethylene-co-vinyl acetate having a vinyl acetate concentration of greater than or equal to about 12 percent by weight. In some embodiments, the top layer 316 can include polyethylene-co-vinyl acetate having a vinyl acetate concentration of less than about 33 percent by weight. In some embodiments, the top layer can include a polyethylene-co-vinyl acetate polymer including from about 12 percent by weight to about 33 percent by weight vinyl acetate.

FIG. 4 is a cross-sectional view of a coating 400 in accordance with another embodiment. In this embodiment, a primer layer 404 (or non-expandable layer) is disposed upon a substrate 402. The primer layer 404 can have a thickness of greater than or equal to about 1 micron. In some embodiments, the primer layer 404 can be from about 1 microns to about 5 microns thick.

An expandable layer 406 (or active agent layer) is disposed upon the primer layer 404. The expandable layer 406 can have a thickness of greater than about 2 microns. In some embodiments the expandable layer 406 can have a thickness of greater than about 5 microns. In some embodiments, the expandable layer 406 can be from about 5 microns to about 100 microns thick.

In this embodiment, a top layer 408 is disposed on the expandable layer 406. The top layer 408 can have a thickness of greater than about 1 micron. In some embodiments, the top layer 408 can be from about 1 micron to about 50 microns thick.

### Substrates

It will be appreciated that embodiments of the invention can be used in conjunction with various types of substrates. Exemplary substrates can include metals, polymers, ceramics, and natural materials. Metals can include, but are not limited to, cobalt, chromium, nickel, titanium, tantalum, iridium, tungsten and alloys such as stainless steel, nitinol or cobalt chromium. Suitable metals can also include the noble metals such as gold, silver, copper, platinum, and alloys including the same.

Substrate polymers include those formed of synthetic polymers, including oligomers, homopolymers, and copolymers resulting from either addition or condensation polymerizations. Examples include, but not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, styrene, vinyl chloride, vinyl acetate, vinyl pyrrolidone, and vinylidene difluoride, condensation polymers including, but are not limited to, polyamides such as polycaprolactam, polylauryl lactam, polyhexamethylene adipamide, and polyhexamethylene dodecanediamide, and also polyurethanes, polycarbonates, polysulfones, poly(ethylene terephthalate), polytetrafluoroethylene, polyethylene, polypropylene, polylactic acid, polyglycolic acid, polysiloxanes(silicones), cellulose, and polyetheretherketone.

Embodiments of the invention can also include the use of ceramics as a substrate. Ceramics include, but are not limited to, silicon nitride, silicon carbide, zirconia, and alumina, as well as glass, silica, and sapphire.

Certain natural materials can also be used as a substrate in some embodiments including human tissue, when used as a component of a device, such as bone, cartilage, skin and enamel; and other organic materials such as wood, cellulose, compressed carbon, rubber, silk, wool, and cotton. Substrates can also include carbon fiber. Substrates can also include resins, polysaccharides, silicon, or silica-based materials, glass, films, gels, and membranes.

### Medical Devices

It will be appreciated that embodiments of the invention can be used in conjunction with, and can include, many different types of medical devices. Embodiments of the invention can include and can be used with both implantable devices and non-implantable medical devices. Embodiments of the invention can include and can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

Classes of non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

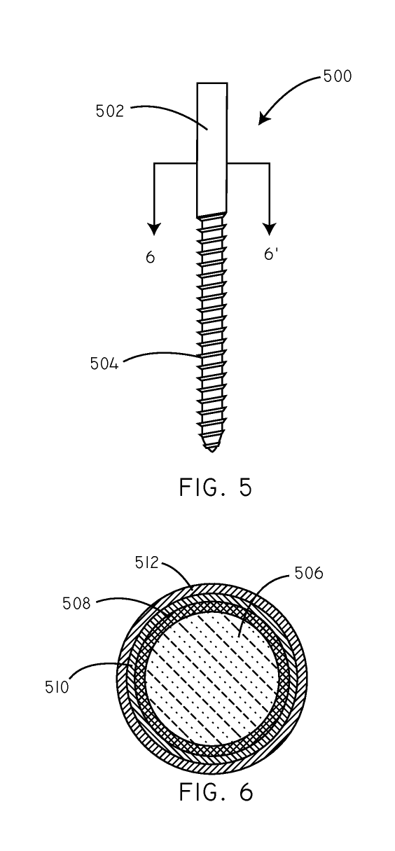

As a specific example, referring now to FIG. 5, a bone screw 500 is shown in accordance with an embodiment herein. The bone screw 500 can include a threaded portion 504 and, optionally, a shank 502. Referring now to FIG. 6 a schematic cross-sectional view is shown of the bone screw as taken along line 6-6′ of FIG. 5. In this embodiment, a primer layer 508 is disposed on top of a substrate 506. The substrate 506 can include, but is not limited to, a metal or a ceramic, such as those described below. Further, an expandable layer 510 including an active agent is disposed on top of the primer layer 508. Various active agents can be used, such as those described above. However, as a specific example, the hydrophilic active agent can include, but is not limited to, antibiotics such as gentamycin and tobramycin, amongst others. The expandable layer 510 can include polymers as described herein. Finally, a top layer 512 is disposed on top of the expandable layer 510. The top layer 512 can be configured to stretch as the expandable layer 510 swells. The top layer 512 can further modulate release of the hydrophilic active agent. The top layer 512 can include polymers that allow the expandable layer to expand such as described herein.

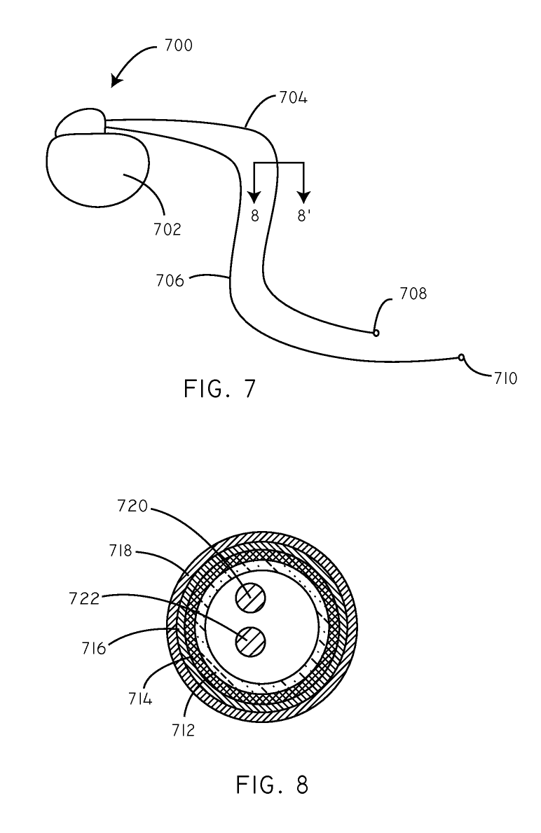

As another example, embodiments herein can be used in conjunction with and/or include electrical stimulation leads, such as cardiac pacing leads. Referring now to FIG. 7, a cardiac pacing system 700 is shown in accordance with an embodiment herein. The cardiac pacing system 700 can include an electrical pulse generator 702 and a pair of leads 704 and 706 to deliver electrical pacing pulses to cardiac tissue. The pacing leads 704, 706 can also include electrodes 708, 710 respectively to interface with cardiac tissue. Referring now to FIG. 8 a schematic cross-sectional view is shown of pacing lead 704 as taken along line 8-8′ of FIG. 7. In this embodiment, a primer layer 714 is disposed on top of a substrate 712 or sheath member. The substrate 712 can include, but is not limited to, polymers such as silicone, polyethylene, and polyurethane. Further, an expandable layer 716 including a hydrophilic active agent is disposed on top of the primer layer 714. The hydrophilic active agent can be, for example, one with anti-inflammatory activity including, but not limited to, dexamethasone phosphate. However, other active agents such as those described above can also be used. The expandable layer 716 can include polymers as described herein. Finally, a top layer 718 is disposed on top of the expandable layer 716. The top layer 718 can be configured to stretch as the expandable layer 716 swells. The top layer 718 can further modulate release of the hydrophilic active agent. The top layer 718 can include polymers as described herein that allow the expandable layer to expand.

In some aspects, embodiments of the invention can include and be utilized in conjunction with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in conjunction with ophthalmic devices configured for placement at an external or internal site of the eye.

Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. No. 6,719,750 B2 (“Devices for Intraocular Drug Delivery,” Varner et al.) and U.S. Pat. No. 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. No. 11/204,195 (filed Aug. 15, 2005, Anderson et al.), Ser. No. 11/204,271 (filed Aug. 15, 2005, Anderson et al.), Ser. No. 11/203,981 (filed Aug. 15, 2005, Anderson et al.), Ser. No. 11/203,879 (filed Aug. 15, 2005, Anderson et al.), Ser. No. 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

The present invention may be better understood with reference to the following examples. These examples are intended to be representative of specific embodiments of the invention, and are not intended as limiting the scope of the invention.

### EXAMPLES

### Example 1

### Fab Elution and Hydration Kinetics of Expandable and Non-Expandable Polymer Coated Intravitreal Implants

Preparation of Spray-Dried Rabbit Fab Particles

Rabbit Fab IgG (Cat. No. 0125-01, SouthernBiotech, Birmingham, Ala.) was used as a model hydrophilic agent. Rabbit Fab was spray-dried (Buchi Mini Spray Dryer B-290) with trehalose resulting in particles containing approximately 70 wt. % protein and 30 wt. % trehalose. The particle size of the spray-dried rabbit Fab, determined by a Sympatech laser diffraction particle size analyzer, was <5 microns with a mean size of approximately 2.5 microns.

Ultrasonic Spray-Coating Process

Coating layers were applied onto a drug delivery platform made of a non-ferrous metal alloy (I-VATION® Intravitreal Implants, SurModics, Inc.), by an ultra-sonic atomization spray coating process. All polymers were dissolved in chloroform at 30 mg/ml. Each implant was weighed prior to coating application. An ultrasonic nozzle (60 KHz ultrasonic nozzle from Sono-Tek, Milton, N.Y.) generated an atomized stream of coating material that was directed at the implants. Implants were dried under nitrogen and weighed to obtain coating weights after each coating layer was applied.

A primer layer of poly-n-butylmethacrylate (PBMA) was applied onto the implants, targeting a coating weight of 150 μg per implant. Next, either an expandable or non-expandable polymer layer containing rabbit Fab particles was applied onto the primer layer (this layer can also be referred to as the base coat). For the expandable polymer layer, two polymers were used separately. One polymer was a poly(ether ester) multiblock copolymer based on poly(ethylene glycol) and polybutylene terephthalate including 45 wt. % poly(ethylene glycol) (M.W.=1000) and 55 wt. % polybutylene terephthalate (POLYACTIVE®). A second polymer was poly(ethylene-co-vinyl-acetate) (PEVA) containing 40 wt % vinyl acetate. PBMA was used for a non-expandable coating. Suspension coating solutions were prepared by weighing rabbit Fab particles into a glass vial and then adding polymer that was dissolved in chloroform. The formulations were subjected to an ultrasonic bath for 5 minutes to ensure a homogenous suspension. The final suspensions contained 30 wt. % rabbit Fab particles and 70 wt. % polymer. The suspensions were spray-coated onto the primer layer of the implants, targeting a coating weight of 1500-2000 μg per implant (or 315-420 μg rabbit Fab per implant). Lastly, a top coat was applied onto the rabbit Fab containing layer. Two different polymers, a multi-block copolymer 50GALA50LA (GA=glycolyic acid, LA=lactic acid, numbers indicate wt. % of each block, SYNBIOSYS®) and PBMA were applied with a targeted coating weight of 500 μg per implant.

In vitro Rabbit Fab Elution

Implants were placed in 1 ml cyrovials to which 1 ml phosphate-buffered saline (PBS, pH 7.4) was added. Vials were incubated static at 37° C. and at various time points PBS was removed and replaced with fresh PBS. Elutions were performed for 1 month.

Rabbit Fab Quantification

Rabbit Fab concentrations of the eluents were determined by performing a tryptophan fluorescence assay. See for example, the techniques described by T. E. Creighton in Proteins: Structures and Molecular Properties, 2nd 15 Ed., W. H. Freeman and Company, 1993. In a 96-well microtiter black plate, 100 μL of eluent samples and a set of serially diluted standards of Fab were added. To all wells, 100 μL 12 N guanidine HCl in deionized water was added. The plate was kept at −20° C. for 10 minutes and fluorescence was measured using a fluorescence microplate reader (λex=290 nm, λem=370 nm). The concentration of Fab in the eluent samples was determined by interpolating fluorescence units from the standard curve.

Hydration Kinetics

After sampling the eluents, prior to dispensing fresh PBS to the vials, implants were blotted dry and weighed by an analytical balance (Mettler Toledo—XS204). The implants were then returned to PBS to continue elution testing. Water uptake data was corrected for the estimated mass loss due to rabbit Fab release.

Results

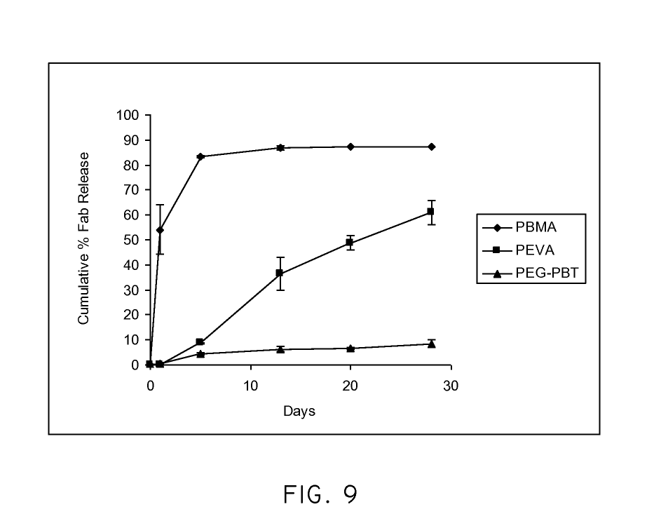

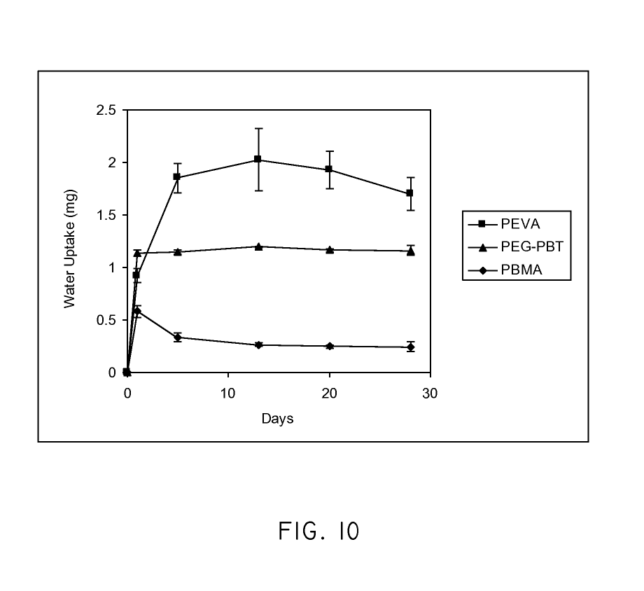

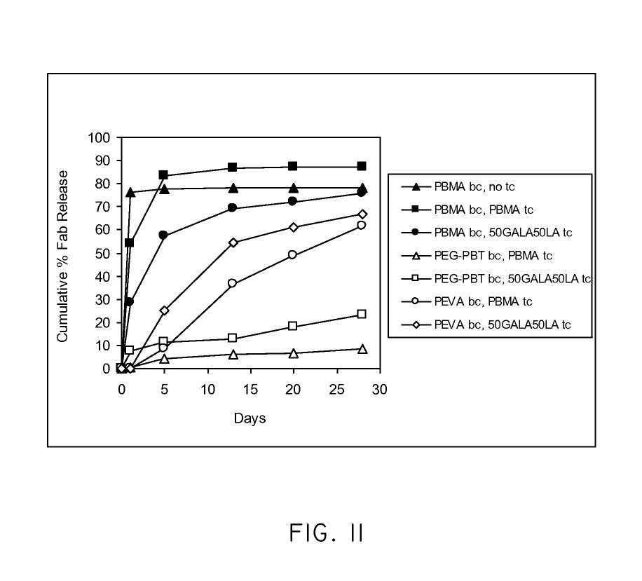

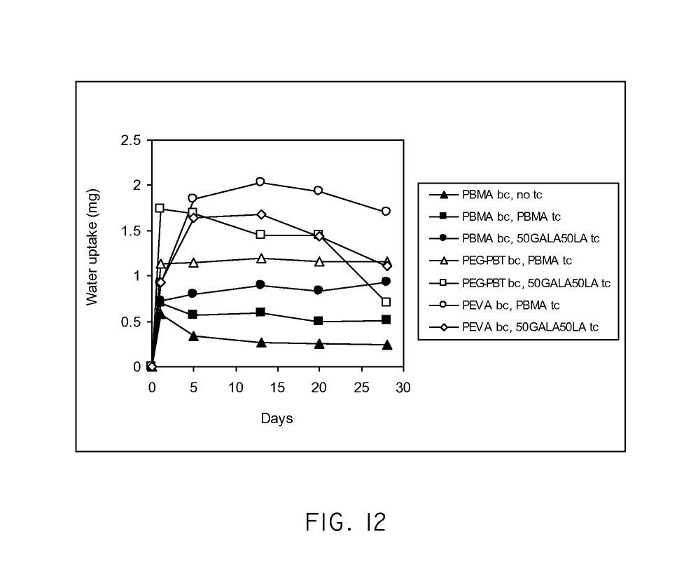

On day 5, the non-expandable polymer coating PBMA released 83% of its rabbit Fab load in contrast to only 9% for the PEVA polymer and 4% for the PEG-PBT polymer (FIG. 9). The PEVA40 and PolyActive coatings absorbed approximately 1.2-2.0 mg of water by 13 days in contrast to the PBMA coating that absorbed approximately 0.5 mg water at its maximum on day 1 (FIG. 10). Cumulative percent Fab release and water uptake results can be seen for non-expandable and expandable polymer coatings with various top coats in FIGS. 11 and 12.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

1. A medical device comprising:
a substrate;
a primer polymer layer disposed on the substrate;
an expandable layer disposed on the primer polymer layer; and
a hydrophilic active agent dispersed within the expandable layer.

2. The medical device of claim 1, further comprising a top coat disposed on the expandable layer, the top coat comprising polyethylene-co-vinyl acetate (PEVA).

3. The medical device of claim 2, the polyethylene-co-vinyl acetate having a vinyl acetate concentration of between about 12 percent and 33 percent by weight.

4. The medical device of claim 1, the expandable layer configured to expand in volume at least about 5% after insertion into a subject.

5. The medical device of claim 2, the expandable layer comprising polyethylene-co-vinyl acetate (PEVA).

6. The medical device of claim 5, the polyethylene-co-vinyl acetate in the expandable layer having a vinyl acetate concentration of greater than or equal to about 33 percent by weight and less than or equal to about 40 percent by weight.

7. The medical device of claim 1, the expandable layer comprising at least one selected from the group consisting of cross-linked polysiloxane (silicone rubber) and butyl rubber.

8. The medical device of claim 1, the expandable layer comprising a hydrogel.

9. The medical device of claim 1, the expandable layer comprising poly lactide-co-glycolide (PLGA).

10. The medical device of claim 1, the expandable layer having a thickness of at least about 5 microns.

11. The medical device of claim 1, the primer layer configured to expand in volume less than about 5% after insertion into a subject.

12. The medical device of claim 1, the primer polymer layer comprising poly-n-butyl methacrylate (PBMA).

13. The medical device of claim 1, the primer polymer layer comprising polystyrene.

14. The medical device of claim 1, the primer polymer layer having a thickness of at least about 1 micron.

15. The medical device of claim 1, the hydrophilic active agent comprising at least one selected from the group consisting of a protein and a nucleic acid.

16. The medical device of claim 1, the expandable layer comprising at least trace amounts of a solvent effective to solvate the primer polymer layer disposed on the substrate.

17. The medical device of claim 1, the substrate comprising a portion of at least one selected from the group consisting of an eye coil, a stent, an electrical stimulation lead, and a bone screw.

18. The medical device of claim 1, the substrate selected from the group consisting of glasses, metals, and ceramics.

19. A method of forming a medical device comprising:
depositing a primer layer onto a substrate, the primer layer comprising a primer polymer; and
depositing an expandable layer onto the primer layer, the expandable layer comprising
a expandable polymer, and
a hydrophilic active agent;
wherein the expandable layer is deposited with a solvent that is effective to solvate both the expandable polymer and the primer polymer.

20. The method of claim 19, further comprising depositing a top coat onto the expandable layer.
